(19) United States
(12) Patent Application Publication
(10) Pub. No.: US 2016/0114048 A1
(43) Pub. Date: Apr. 28, 2016

(54) THERMOLABILE DRUG RELEASE FORMULATION

(71) Applicant: UNIVERSITEIT ANTWERPEN, Antwerpen (BE)

(72) Inventors: Arnout Everaert, Wilrijk (BE); Annick Ludwig, Wilrijk (BE); Dave Van Den Plas, Wilrijk (BE); Wim Weyenberg, Wilrijk (BE)

(73) Assignee: Universiteit Antwerpen, Antwerpen (BE)

(21) Appl. No.: 14/891,065

(22) PCT Filed: May 14, 2014
(86) PCT No.: PCT/EP2014/059843

§ 371 Date: Nov. 13, 2015

(30) Foreign Application Priority Data
May 16, 2013 (EP) 13167977.1

(51) Int. Cl. A61K 47/38; A61K 38/38; A61K 31/352; A61K 38/47; A61K 9/00; A61K 47/26

(52) U.S. Cl. CPC A61K 47/38; A61K 9/0048; A61K 47/26; A61K 31/352; A61K 38/47; A61K 38/38; C12Y 302/01017

(57) ABSTRACT

The present invention relates to a drug release formulation, in particular a sustained release formulation for ophthalmic applications and a method of preparing same. The method is based on the hydration of a given solid polymeric matrix material under mild conditions, allowing versatility with respect to the drug to be formulated. Both said solid polymeric matrix material as well the API hydrated formulation is an object of the present invention. The thus obtained material is particularly suitable for prolonged and sustained delivery of medication to the eye. Thus in a further aspect, the present invention provides the use of said solid polymeric matrix material as well the API hydrated formulation, in ophthalmic applications.

### FIELD OF THE INVENTION

The present invention relates to a method for manufacturing a polymeric matrix carrier system for (thermo)-labile drugs. Using the methods of the present invention, it becomes possible to charge (thermo)-labile molecules in a hydrogel carrier in order to obtain sustained release formulations of such (thermo)-labile drugs. Thus in a further aspect the present invention provides such drug release formulation, in particular a sustained release formulation for ophthalmic applications and a method of preparing same. The method is based on the hydration of a given solid polymeric matrix material under mild conditions, allowing versatility with respect to the drug to be formulated. Both said solid polymeric matrix material as well the Active Pharmaceutical Ingredient (API) hydrated formulation is an object of the present invention. The thus obtained material is particularly suitable for prolonged and sustained delivery of medication to the eye. Thus in a further aspect, the present invention provides the use of said solid polymeric matrix material as well the API hydrated formulation, in ophthalmic applications.

### BACKGROUND TO THE INVENTION

The present invention is directed to a method of manufacturing a polymeric matrix suitable for loading an API under mild conditions. It further provides loading of a drug into said polymeric matrix under mild conditions with the objective to obtain a drug release formulation, in particular a sustained drug release formulation. The method and polymeric carrier system thus obtained, is particularly suitable for ophthalmic articles. Ophthalmic articles typically consist of organic polymeric or co-polymeric matrixes, and there are currently a plurality of methods to incorporate drugs into said material. Within said methods two main categories can be recognized.

In a first category, also referred to as cast molding, the drugs are added to the pre-polymerization mixture comprising the reagents like monomers, co-monomers, solvent and initiators, to make said organic polymeric or co-polymeric matrixes. After the polymerization reaction the drugs will be entrapped into the ophthalmic article. Such protocol is for example disclosed in the International patent publication WO9405257. In said reference the drug is dissolved in a plasticizer solution, and subsequently blended with the polymeric carrier components Eudragit S100, and Methocel J4. For the polymerization reaction this blend is added to a melt extruder at high temperatures above 160° C. and kept at 160° C. to extrude rods comprising the initially dissolved drug. In WO2002074196 the therapeutic agent is premixed in a solution of hydroxypropyl methylcellulose, and subsequently brought in contact with the initiator consisting of superhydrolized polyvinyl alcohol under intense stirring. In either of said manufacturing methods, the drug or therapeutic agent is present in the reaction mixture during the polymerization reaction and accordingly exposed to the non-mild and non-ambient reaction conditions.

In a second category, also referred to as impregnation, the matrix is immersed in a solution containing the drug, allowing diffusion of the latter into the matrix. However, in order to realize mass transfer of the drug from the solution into the matrix, this process requires facilitators like thermal transfer, the presence of impregnation additives, the application under pressure, or combinations thereof.

Evidently, in each of the foregoing categories the reaction conditions, such as the aforementioned high temperatures (also in case of cast molding), the presence of additives, the application under pressure, may irreversibly degrade the drug(s) to be loaded into the ophthalmic material. It is accordingly an object of the present invention to provide a method of loading additives into an ophthalmic material under mild, ambient conditions.

### BRIEF DESCRIPTION OF THE DRAWINGS

FIG. 1: General Scheme in the manufacture of drug loaded HPMC polymeric matrix, applying the methods of the present invention.

FIG. 2 A: Water absorption of a 150 mg drug-loaded insert having 20% wt of HPMC type E10M and 10% wt glycerol. B: Water absorption of a 150 mg drug-loaded insert having 20% wt of HPMC type K100M and 5% wt glycerol.

FIG. 3: Cumulative release profile of lysozyme and sodium fluorescein loaded inserts from two types (E10M and K100M) of HPMC inserts. Each of said HPMCs tested at two different end concentrations of 20% wt and 15% wt, respectively.

FIG. 4: Peppas-Korsmeyer plot for the kinetics of the lysozyme release of two types (E10M (A) and K100M (B)) of HPMC inserts at HPMC concentrations of 20% (w/w).

FIG. 5: Peppas-Korsmeyer plot for the kinetics of the lysozyme release of two types (E10M (A) and K100M (B)) of HPMC inserts at HPMC concentrations of 15% (w/w).

FIG. 6: Cumulative release profile of lysozyme and sodium fluorescein loaded inserts from E10M HPMC blister unit inserts (n=8).

FIG. 7: Cumulative release of albumin from inserts prepared by 50% or 100% dehydration, and 24 h or 72 h charging. (n=8).

### SUMMARY OF THE INVENTION

In a first aspect the present invention is directed to a method of manufacturing a drug-loaded carrier suitable for ophthalmic applications, said method comprising the steps of;
preparing a HPMC polymer using standard conditions, but in absence of the drug of interest;
dehydrating the thus obtained HPMC polymer to yield a partly dehydrated or dehydrated HPMC polymer; and
contacting said partly dehydrated or dehydrated HPMC polymer with a rehydration solution comprising the drug of interest, (in particular under mild, ambient conditions) to obtain said drug loaded carrier.

As evident from the accompanying examples, the HPMC polymers are prepared using standard conditions, e.g. by stirring a suspension of the HPMC particles in a suitable solvent (typically water) at temperatures above the gelation temperature, but below the boiling point of the suspension (typically in the range of 60-100° C.). Applying such shear forces (stirring) at these elevated temperatures results in a homogenous distribution of the HPMC particles in the solvent. Upon subsequent cooling of the suspension, the HPMC particles dissolve with the formation of hydrogen bounds between the HPMC molecules and the solvent, yielding a colloidal solution, in the art also known as a HPMC hydrogel. In other words, within the context of the present invention, ‘HPMC polymer’ or ‘HPMC polymers’ refers to a colloidal solution of HPMC in a suitable polar protic solvent, such as for example formic acid, ethanol, methanol, acetic acid and water. Also known as a HPMC hydrogel. In a particular embodiment, the HPMC polymers are prepared as an aqueous solution comprising one or more of the HPMC's as herein provided, in a final concentration of about 15%-25% wt, more in particular a HPMC content of about 20% wt, or 15% wt, with subsequent maturation in a refrigerator (2° C. for at least 2 hours).

The methods in the manufacture of a drug carrier (i.e. a polymeric drug carrier) according to the present invention are characterized in that the HPMC polymers are prepared in the absence of the drug of interest and in the presence of a dehydration step of the HPMC polymers thus obtained. As further detailed below, dehydration of the HPMC polymer generally refers to the removal of the solvent (in particular water) from the HPMC hydrogel. Dehydration is either complete (full removal of the solvent) or only partially, i.e. to a desired degree of dehydration, typically towards HPMC concentrations starting at 25% wt. The dehydrated HPMC polymers are subsequently loaded with the drug of interest in a rehydration step, i.e. using a rehydration solution comprising the drug of interest. It will be apparent to the skilled artisan, that the intermediate product, i.e. the partially dehydrated or dehydrated HPMC polymer in the aforementioned manufacturing process, could be used and commercialized as a starting material in the manufacture of a drug loaded HPMC polymeric matrix, and accordingly referred to as a drug carrier, in particular suitable for ophthalmic applications.

Thus in a second aspect the present invention is directed to a drug carrier suitable for ophthalmic applications, consisting of a partly dehydrated or dehydrated hydroxypropyl methyl cellulose (HPMC) polymer wherein the fully dehydrated carrier comprises up to 100% wt of said HMPC. Simply consisting of a partly dehydrated or fully dehydrated HPMC (commonly referred to as ‘dehydrated HPMC’ or ‘dehydrated HPMC polymer’—infra), the drug carrier as used herein, is to be understood as being a starting material in the preparation of a ‘drug loaded’ polymeric matrix. Compared to the prior art methods wherein the drug is present during the polymerization reaction of the matrix, the drug carrier of the present invention is prepared in the absence of the drug of interest. As such, the drug carrier of the present invention consists of a partly dehydrated or a dehydrated hydroxypropyl methyl cellulose (HPMC) polymer wherein the partly dehydrated or dehydrated carrier comprises from 25% wt up to 100% wt of said HPMC, and characterized in that it does not comprise a drug of interest. Such partly dehydrated of dehydrated HPMC drug carrier is particularly suitable for loading under mild conditions. It is accordingly an aspect of the present invention to provide the use of such dehydrated or partly dehydrated hydroxypropyl methyl cellulose (HPMC) polymer, as a drug carrier suitable for ophthalmic applications; or in the manufacture of a drug loaded polymeric matrix.

The higher the dehydration grade of the HPMC, the more drug can be loaded during the rehydration step, in the methods according to the present invention. For the avoidance of doubt, as used herein the hydration step is not limited to the use of an aqueous solutions, but generally refers to the charging of the dehydrated carrier with a suspension comprising the drug of interest. After loading the drug carrier desirably has a HPMC content in the range of about 10%-30% wt, in particular in the range of about 15%-25% wt, more in particular a HPMC content of about 20% wt. In a particular embodiment the drug carrier has a HPMC content of about 25% wt after loading. As such in principle any HPMC polymer that has been dehydrated to a HMPC content above any of the foregoing concentrations, can be used in the present invention. In the exemplified embodiments partly dehydrated HPMC with HPMC concentrations starting at 25% wt; in particular starting at 30% wt; more in particular starting at 35% wt; even more in particular starting at 45% wt were shown useful in the methods of the present invention. Thus in a further embodiment of the present invention the drug carrier consists of a partly dehydrated HPMC, wherein the carrier comprises from 25% wt up to 100% wt of said HPMC; in particular from 30% wt up to 100% wt of said HPMC.

In principle any type of HPMC polymer can be used in the foregoing application. In one embodiment of the present invention, the HPMC is selected from the group consisting of E-type, F-type, K-type or combinations thereof. As the number of hydroxyl-groups present influences rehydration of the HPMC, better results are obtained with E-type and K-type. These latter HPMC types have a higher viscosity when compared to the F-type, and as such a lower degree of dehydration is required when applying it as a drug carrier in the context of the present invention. As the degree of dehydration is one of the factors influencing the drug loading step, one preferably starts with a partly dehydrated HPMC drug carrier wherein the degree of dehydration is less and closer to the lower values of the aforementioned ranges, in particular when subsequently loaded with low molecular weight molecules. In case of large molecular weight molecules such as proteins, the degree of dehydration is preferably closer to the higher values of the aforementioned ranges. Thus in a further embodiment the HPMC is selected from E-type, K-type or combinations thereof. In one embodiment the drug carrier accordingly consists of a partly dehydrated HPMC, wherein said HPMC is selected from E-type, K-type or combinations thereof, and wherein the carrier comprises 25% wt up to 100% wt of said E-type, K-type or combinations thereof; in particular from 30% wt up to 100% wt of said E-type, K-type or combinations thereof.

As used herein ‘dehydrated HPMC’ or ‘dehydrated HPMC polymer’ corresponds to the removal of the solvent (in particular water) from the HPMC up to its maximum being equivalent to a constant weight attained when dried. As such in the dehydrated HPMC polymer and upon removal of all the solvent, the material would consist solely of the HPMC polymer carrier, thus attaining a HPMC concentration of 100% wt. The dehydration may take place at any temperature at which water molecules can be removed from the hydrogel. Without intention of being complete, and without being limited thereto, the following embodiments provide possible configurations under which dehydration of the HPMC polymer can be achieved. In one embodiment the dehydration is performed at temperatures below the gelation temperature (T_{gel}) of the HPMC polymer. In said instance water evaporates from the hydrogel, and the latter takes the shape of its receptacle (the hydrogel “drops in” and takes form in container). Above the T_{gel} a strong hydrophobic interaction between the HPMC molecules is created and hydrogen bonds between water molecules and HPMC are broken. As a consequence, under these other dehydration conditions, a phase separation occurs and the HPMC polymer is equally decreased in all directions while maintaining the geometrical shape. Either dehydration method can be used in the context of the present invention. It has been observed that partly dehydrated or dehydrated HPMC polymers obtained at temperatures above T_{gel}, are more homogeneously loaded with the drug of interest during the dehydration step. Thus in one embodiment the HPMC polymer is dehydrated by exposing it to temperatures above T_{gel}, for a time sufficient to attain the desired degree of dehydration (herein below expressed as the attained concentration of HPMC polymer and starting at 35% wt of the HPMC). Assessing whether the desired degree of dehydration is being achieved, can be done by measuring the weight loss (loss of solvent (in particular water)) of the HPMC polymer, where the HMPC is fully dehydrated when a constant weight is attained when drying.

In one embodiment the dehydrated hydroxypropyl methyl cellulose (HPMC) polymer used in the drug carrier consists of E-type HPMC, K-type HPMC, or combinations thereof and the carrier comprises at least 35% wt of said HPMC(s). In another embodiment the dehydrated hydroxypropyl methyl cellulose (HPMC) polymer used in the drug carrier consists of E-type HPMC, J-type, K-type HPMC, or combinations thereof, and wherein the carrier comprises at least 30% wt, at least 45%, at least 50% wt, or at least 75% wt of said HMPC. In one embodiment the dehydrated hydroxypropyl methyl cellulose (HPMC) polymer used in the drug carrier consists of E-type HPMC or K-type HPMC and the carrier comprises at least 35% wt of said HPMC. In another embodiment the dehydrated hydroxypropyl methyl cellulose (HPMC) polymer used in the drug carrier consists of E-type HPMC or K-type HPMC and the carrier comprises at least 45% wt of said HPMC. In a particular embodiment the dehydrated hydroxypropyl methyl cellulose (HPMC) polymer used in the drug carrier consists of E-type HPMC or K-type HPMC and the carrier comprises at least 30% wt of said HMPC. In a particular embodiment the dehydrated hydroxypropyl methyl cellulose (HPMC) polymer used in the drug carrier consists of E-type HPMC and the carrier comprises at least 50% wt of said HMPC. In a particular embodiment the dehydrated hydroxypropyl methyl cellulose (HPMC) polymer used in the drug carrier consists of E-type HPMC and the carrier comprises at least 75% wt of said HMPC. In a particular embodiment the dehydrated hydroxypropyl methyl cellulose (HPMC) polymer used in the drug carrier consists of E-type HPMC and the carrier comprises at least 35% wt of said HMPC. In a particular embodiment the dehydrated hydroxypropyl methyl cellulose (HPMC) polymer used in the drug carrier consists of E-type HPMC and the carrier comprises at least 50% wt of said HMPC. In a particular embodiment the dehydrated hydroxypropyl methyl cellulose (HPMC) polymer used in the drug carrier consists of E-type HPMC and the carrier comprises at least 75% wt of said HMPC. In a particular embodiment the dehydrated hydroxypropyl methyl cellulose (HPMC) polymer used in the drug carrier consists of E-type HPMC and the carrier comprises about 100% wt of said HMPC. In another particular embodiment the dehydrated hydroxypropyl methyl cellulose (HPMC) polymer used in the drug carrier consists of K-type HPMC, and wherein the carrier comprises at least 45% wt of said K-type HMPC.

In a second aspect the present invention provides a method for loading the aforementioned HPMC carriers with a drug of interest, said method comprising exposing the drug carrier according to any one of the foregoing embodiments, to a hydration solution comprising said drug and a solvent (preferably water); in particular for a time sufficient to allow complete absorption of the hydration solution. As the hydration step is preferably performed at a temperature close to freezing temperature and up to room temperature, in particular at about 4, 5, 6, 7, 8, 9, or 10° C., dynamics are slow and this hydration step may take up to one or more weeks. As evident from the examples hereinafter, when prepared as blister package units, the rehydration and loading of the dehydrated hydroxypropyl methyl cellulose (HPMC) polymers can be realized in incubation times as short as a couple of days and even after couple of hours (1, 2, 3, 4, 5 or more) the blisters can be sealed, allowing the rehydration to continue in the closed package. For partly dehydrated HPMC blister package units of the present invention, characterized in that the HPMC content of said partly dehydrated HPMC blister package units is within the range of 25% wt up to 100% wt, the rehydration and loading step can even be achieved in times as short as a couple of minutes (2, 3, 4, 5, 6, 7, 8, 9, 10, 15, 20, 25 or more) In addition, when prepared as blister package units, dehydration of the unloaded HPMC polymer can simply be achieved through air drying at temperatures below the gelation temperature of the HPMC polymer, more in particular by air drying at room temperature.

Thus in one embodiment of the present invention the method of manufacturing a drug loaded polymeric matrix suitable for loading under mild conditions comprises the steps of;
preparing a HPMC polymer dispersion using standard conditions;
pouring said HPMC polymer dispersion into blister package units;
dehydrating the thus obtained HPMC polymer blister package units to yield partly dehydrated or dehydrated HPMC polymer blister package units;
contacting said partly dehydrated or dehydrated HPMC polymer blister package units under mild, ambient conditions, with a rehydration solution comprising the drug of interest; and
sealing the blister package.

In this embodiment the HPMC polymer could be any one as herein described, in particular the HPMC polymer use in this manufacture process consists of E-type HPMC, more in particular E-type HPMC selected from E4M Premium, or E10M Premium CR. In this method dehydration of the HPMC polymer can be realized using any one of the dehydration protocols herein described. For the HPMC polymer blister package units, the usable dehydration temperature may be determined by the operational temperature range of the blister package. Using for example PVC blisters, maximum usable temperatures can be as low as about 60° C. For polyvinylidenechloride (PVDC), the upper working temperature is in the range of 80-100° C.; and when using cyclic olefin copolymer (COC) as blister packaging material the working range even extends from 80-120° C. In one embodiment the dehydration step used for the HPMC polymer blister package units, is air drying of the HPMC polymer blister package units below the T_{gel}; in particular at room temperature for a time sufficient to achieve the desired degree of dehydration.

In principle any art known blister packaging material can be used, and based its product specifications the skilled artisan would know which to choose taking into account the T_{gel} of the HPMC polymer being used. In one embodiment the blister package is selected from the group comprising PVC, PVDC or COC.

Within this method, the dehydrated HPMC polymer blister package units are loaded with the drug of interest by bringing them into contact with the rehydration solution. Again any of the hydration solutions herein described can be used in this embodiment. One of such hydration solutions, observed to be particularly suitable in case the HPMC polymer is fully dehydrated is characterized in comprising HPMC. When present, the amount of HPMC in the rehydration solution should be such that the solutions remains pourable, i.e. up to a viscosity of about and between 3300±50 Pa.s. to 3650±50 Pa.s. as determined at 20° C., gap 0.5 mm size, a constant shear rate 0.1 s−1 on an Anton Paar rheometer (MCR102) with a PP20 (parallel plate) 20 mm diameter; or up to a viscosity corresponding to the viscosity determined for an HPMC solution of 8% wt of E10M at 20° C., gap 0.5 mm size, a constant shear rate 0.1 s−1 on an Anton Paar rheometer (MCR102) with a PP20 (parallel plate) 20 mm diameter. In one embodiment, comprising HPMC in the range of between and about 1 to 15% wt; in particular in the range of between and about 1 to 10% wt; more in particular in the range of between and about 1 to 5%, 6%, 7%, 8% or 9% wt; even more in particular in the range of between and about 3 to 8% wt of HPMC. The presence of a small amount of HPMC in the hydration solution results in a more homogenous distribution of the drug of interest within the HPMC polymer, irrespective of the dehydration protocol being used. This effect is more pronounced for high molecular weight drugs, such as proteins. In principle any of the proposed HPMC's can be used, but preferably the same or the same combination as used in the manufacture of the HPMC hydrogel.

In one embodiment of the present invention the method is further characterized in that the hydration solution comprises a plasticizer. In a particular embodiment said plasticizer is present in said hydration solution in an amount up to 50% wt; more in particular the plasticizer is present in said hydration solution in an amount up to about 10% wt, in particular in an amount of between and about 1% wt to 10% wt, more in particular 5% wt to 10% wt; even more particular in an amount of between and about 1% wt to 5% wt.

Suitable plasticizers include polyethylene glycols (PEGs), such as PEG 400 and PEG 1000; glycerol, and sorbitol; in particular selected from glycerol and sorbitol; even more in particular consisting of glycerol.

Thus in a particular embodiment of the present invention, the plasticizer used in the method for loading the carrier with a drug, is glycerol and said plasticizer is present in said hydration solution in amount of between and about 5% wt to 10% wt.

As it is an object of the present invention to provide a method for the preparation of a carrier for the loading a material under mild conditions, it should not come to a surprise that the foregoing method in its different embodiments can be performed at atmospheric pressure and temperatures up to and below room temperature. Different from the known impregnation methods, and as evident from the examples hereinafter, in the present instance the rehydration and loading step is performed at atmospheric pressure and at a temperature in a range from 0° C. up to room temperature. In particular in a temperature range from about 0° C. to about 25° C. Using such mild rehydration conditions, the risks of irreversibly damaging the drug(s) to be loaded into the HPMC polymeric material, are avoided. As such, this new drug carrier is particularly useful as carrier for temperature and/or pressure sensitive drugs, and dependent on the shaping method being employed also for drugs being sensitive to degradation when exposed to shear forces and/or entrapped air. As evident from the examples, the carrier of the present invention thus even allows peptides and/or proteins to be incorporated. Using the carriers as described herein, such sensitive drugs, including peptides and/or proteins, can be processed at atmospheric pressure, low temperatures (in particular just above freezing point), without exposure to shear forces, without the entrapment of air, and without air drying, eventually at an elevated temperature, all of which could lead to oxidation and degradation of the peptides and/or proteins.

In a preferred embodiment the methods of the present invention are used in the manufacture of an ophthalmic drug carrier. It is thus a further object of the present invention to provide an ophthalmic drug carrier manufactured using the methods of the present invention.

After loading the carrier with the drug, the thus drug loaded material may be further processed, in particular to prepare therapeutic ophthalmic articles. Such ophthalmic articles are typically in dimensions and shape to allow introduction in the cul-de-sac of the eye. In general these particles may have a shape that can be described as rod-like, disc-like, block-shaped, elongated, football-shaped, rectangular-shaped, half-cylinder-shaped or semi-cylinder shaped and the like. Shaping of the drug loaded material is done using standard procedures, including extrusion or machining. For example, the drug loaded material may be extruded with an extrusion apparatus under mild conditions. Irrespective of the extrusion methods being used, entrapment of air in the drug loaded carrier is best avoided. The presence of air may affect the long term stability and homogeneity of the drug loaded material, in particular when peptides and/or proteins have been incorporated.

Accordingly, in a further embodiment the foregoing method in its different embodiments, may further comprise the step of shaping the drug loaded carrier, such as for example using pressure molding or cold extrusion. In case of ophthalmic applications the drug loaded carrier is for example extruded in rod-like, disc-like, block-shaped, elongated, football-shaped, rectangular-shaped, half-cylinder-shaped or semi-cylinder shaped extrudates; in particular into rod shaped extrudates, suitable for use as ophthalmic inserts.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The invention will now be illustrated by means of the following synthetic and biological examples, which do not limit the scope of the invention in any way.

### EXAMPLES

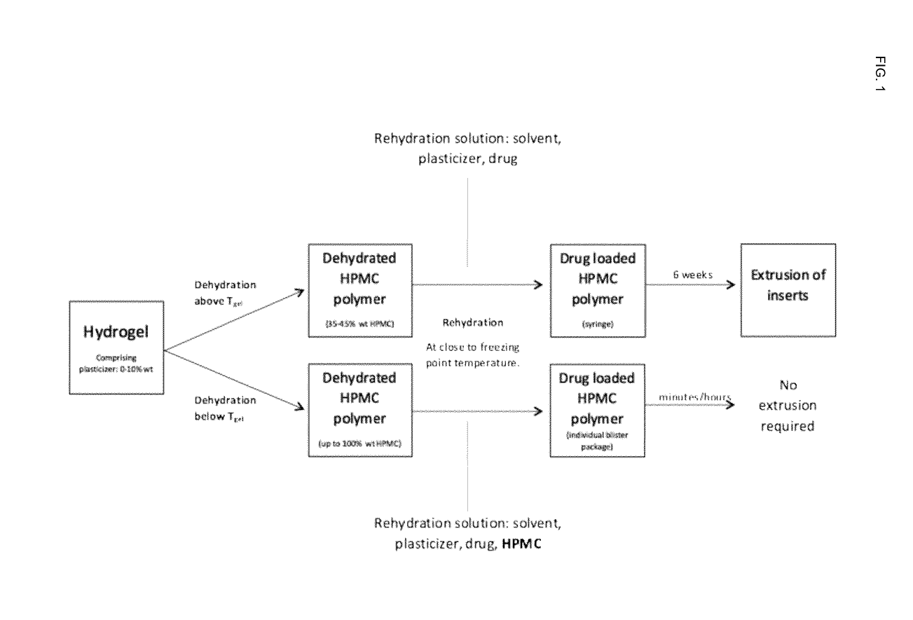

FIG. 1 provides a schematic overview of the new method of the present invention in the manufacture of drug loaded HPMC polymeric matrices. Independent of the two alternative embodiments, these methods comprise;
the manufacturing of a HPMC hydrogel
a dehydration step to achieve dehydrated HPMC polymer; and
a rehydration step to achieve a drug loaded HPMC polymer.

The HPMC hydrogel is prepared under standard conditions, and typically departs from a suspension of HPMC particles in water, optionally comprising a plasticizer in concentrations up to about 10% wt.

The dehydration step is either performed at temperatures above or below the gelation temperature (T_{gel}) of the HPMC polymer. In case of a final extrusion step to achieve the unit dosage forms, the dehydration step is preferably performed at a temperature above the T_{gel} (supra) given an equidimensional shrinkage of the HPMC hydrogel, shown to yield a more homogenous loading of the dehydrated HPMC with the drug of interest. In case the drug loaded HPMC polymeric matrices are directly prepared as unit dosage forms (individual blister package units), the dehydration step is preferably performed at a temperature below the T_{gel} (supra), given the nature of the blister package material. The degree of dehydration, expressed as the final HPMC concentration in the dehydrated hydrogel, typically ranges from between and about 35-45% wt HPMC up to about 100% wt HPMC. For the individual dosage forms the dehydration is preferably up to the higher HPMC concentrations (from about 75% up to 100% wt HPMC), where for the bulk preparation the lower values (from about 35% up to 45% wt HPMC) are more preferred.

In the rehydration step, or the drug loading step, the dehydrated HPMC polymers are brought in contact with a rehydration solution comprising a suitable solvent (preferably water) and the drug of interest. Optionally further comprising a plasticizer in concentrations up to about 10% wt. In one embodiment, the plasticizer is either present in the HPMC suspension used for the manufacture of the HPMC polymer or in the dehydration solution, i.e. in an amount up to about 10% wt, in particular in an amount of between and about 1% to 10% wt, more in particular 5% to 10% wt; even more particular in an amount of between and about 1% to 5% w. In one embodiment, there is no plasticizer in the HPMC suspension used for the manufacture of the HPMC polymer and up to about 10% wt, in particular in an amount of between and about 1% to 10% wt, more in particular 5% to 10% wt; even more particular in an amount of between and about 1% to 5% wt; in the rehydration solution. In another embodiment, there is up to about 10% wt, in particular in an amount of between and about 1% to 10% wt, more in particular 5% to 10% wt; even more particular in an amount of between and about 1% to 5% wt of a plasticizer present in the HPMC suspension used for the manufacture of the HPMC polymer; and no plasticizer in the rehydration solution.

In an even further embodiment, and in particular when applied on unit dosage forms, the rehydration solution may further comprise low amounts of HPMC (supra). The rehydration step is preferably performed at temperatures close to freezing point temperature, but may be as high as about room temperature under atmospheric pressure, i.e. at ambient and mild reaction conditions. Dependent on the size of the dehydrated HPMC polymers the rehydration step may take from a couple of minutes up to a number of weeks. The absence of the further extrusion step, and the much shorter rehydration time required to achieve full loading of HPMC unit dosage forms, the latter has obvious advantages over the bulk approach (infra). Unexpectedly, the presence of low amounts of HPMC in the rehydration solution has a significant impact on the release profile of the drug loaded HPMC polymers, and allows fast loading with delayed release of large molecules, such as proteins (albumin in example 2 below) from the drug loaded inserts.

### Example 1

### Extrusion of Drug Loaded Polymeric Matrices

### 1.A. Methods

The influence of the chemical structure, the molecular weight and the degree of substitution of the polymers used and the type and concentration of plasticizer to the properties of the inserts has been examined.

### 1.1.1. Choice Polymers and Plasticizer

Different polymers in different concentrations were evaluated: hydroxypropyl methyl cellulose (HPMC), hydroxypropyl cellulose (HPC), methyl cellulose (MC), hydroxyethyl cellulose (HEC), Na carboxymethyl cellulose (NaCMC), Na alginate, and carbomer. As eye-compatible plasticizers sorbitol and glycerol were tested in concentrations of 0 to 50% wt.

The only polymers that are eligible to prepare flexible carrier systems having the requisite properties for ophthalmic use to obtain the cellulose derivatives are, in particular, HPMC polymers. In order to obtain flexible drug loaded extrudates, it is important for the polymer that the shrinkage upon dehydration is equal in all directions. Only under said circumstances, the dehydrated material will retain its shape upon hydration with a drug loaded solution. Testing each of the foregoing polymers, it has been observed that for the tested polymers this can only be achieved when using HPMC. Evidently, this observation should not limit the present invention to HPMC as the sole dehydrated polymer that can be used in the context of the present invention, any suitable polymer showing the aforementioned shrinkage behavior can be used as an alternative.

As already mentioned herein before, the type of HPMC, and in particular the ratio of hydroxypropyl- to methyl-cellulose influences the viscosity of the HPMC. The higher the viscosity of the HPMC, less dehydration is required in applying the drug carrier in the methods of the present invention. Better results are obtained with E-type, K-type, and combinations thereof. In the further results one or more of the following materials were specifically used;
K4M Premium, K15M Premium CR, K100M Premium (Colorcon Ltd, UK)
E4M Premium, E10M Premium CR, F4M Premium (Colorcon Ltd, UK)

The HPMC polymers were prepared using standard conditions by stirring at temperatures in the range of 60-100° C. in an aqueous solution comprising one or more of the foregoing HPMC's in a final concentration of about 15%-25% wt, more in particular a HPMC content of about 20% wt, or 15% wt, with subsequent maturation in a refrigerator (2° C. for at least 2 hours)

For the E-type and K-type HPMC polymers in the following examples, the polymers were prepared by stirring at a 90° C. in an aqueous solution comprising up to 15% wt K-Type HPMC and/or up to 25% wt of E-Type HPMC for up to about 5 min. with subsequent maturation in a refrigerator (2° C. for at least 2 hours)

### 1.1.2. Manufacturing Process

The thus obtained HPMC polymers, unloaded with API, were subsequently dehydrated by drying at a temperature above the gelation temperature and below the glass transition temperature Tg (tg) of said HPMC material. In principle the HPMC will be dried up to constant weight under said circumstances, but materials dehydrated up to 100% wt of said HMPC of the original weight can be used as drug carrier in the context of the present invention. For the E-type HPMC and K-Type HPMC materials mentioned above, the polymers were dried to 35% wt and to 45% wt of the original weight. For the E-type HPMC this was realized by drying the material at a temperature of 100° C. for 8 hours. For the K-type HPMC this was realized by drying at a temperature of 150° C. for 8 hours. The dehydrated materials were allowed to cool down, and can be stored for later loading in the refrigerator.

The dehydrated HPMC polymer is subsequently hydrated with a drug containing solution. In the present examples, the drug containing solutions are aqueous solutions either comprising 0.2% wt of Sodium Fluorescein and between 5% to 10% wt of glycerol, or 0.03% wt of lysozyme and 5% wt of glycerol. Hydration is performed by incubating the dehydrated HPMC polymer in said solution at ambient conditions (atmospheric pressure and temperature of just above freezing point up to room temperature) for a time sufficient to allow complete rehydration of the dehydrated HPMC polymer. In the present instance, the dehydrated HPMC polymers were loaded in the refrigerator.

In a final step, the drug loaded and hydrated HPMC's are extruded into clear, homogenous, flexible eye inserts.

### 1.1.3. Determination of the Characteristics of the Drug Loaded Inserts

### 1.1.3.1. Homogeneity

To determine the homogeneity of the drug-loaded inserts, at regular intervals 10 samples of 150 mg where weighed during the extrusion process. The concentration of added drug was determined spectrophotometrically.

### 1.1.3.2. Water Absorption

A quantity of drug-loaded inserts (samples of 150 mg) were weighed and put on the glass filter of the Baumann apparatus. A PBS solution with a pH of 7.4 is used as medium. The mass of the swollen matrix is determined after 60, 120, 240, and 1440 min in order to calculate the water absorption.

### 1.1.3.3. Release Kinetics

Release of the Sodium Fluorescein loaded inserts was determined as follows. After extrusion, a sample of 150 mg of the drug loaded insert was added to 10 ml of a PBS solution (pH 7.4) and the tube incubated in a non-oscillating Hot Water Bad (at 32° C.) to follow sink conditions. After 20, 40, 60, 180, 300 and 1440 min the solution was gently homogenized and 5 ml was pipetted out of the test tube and replaced with 5 ml of fresh PBS-diluted solution. The concentration of Sodium Fluorescein in the 5 ml samples was determined using a UV-VIS spectrophotometer at a wavelength of 484 nm.

For the lysozyme loaded inserts the same protocol was use, but instead of 10 ml of a PBS solution, only 5 ml was added and instead of 5 ml samples, 1 ml samples were taken during the incubation. Also different from the Sodium Fluorescein samples, lysozyme was detected using a UV-VIS spectrophotometer at a wavelength of 280 nm.

The calculated concentrations were plotted as % cumulative release in relation to the measurement after 24 hours. After 24 hours the insert is completely eroded and the farmacon/drug is fully released. For example: this is exactly one of the desired characteristics of an ophthalmic drug formulation. When the material is such that it slowly degrades and disappears when applied in the cul-de-sac of the eye, there is no need to remove it once the drug has been released.

### 1.B. Results

### 1.2.1. Homogeneity

All drug loaded inserts were found to comply with the requirements for ophthalmic applications.

### 1.2.2. Water Absorption

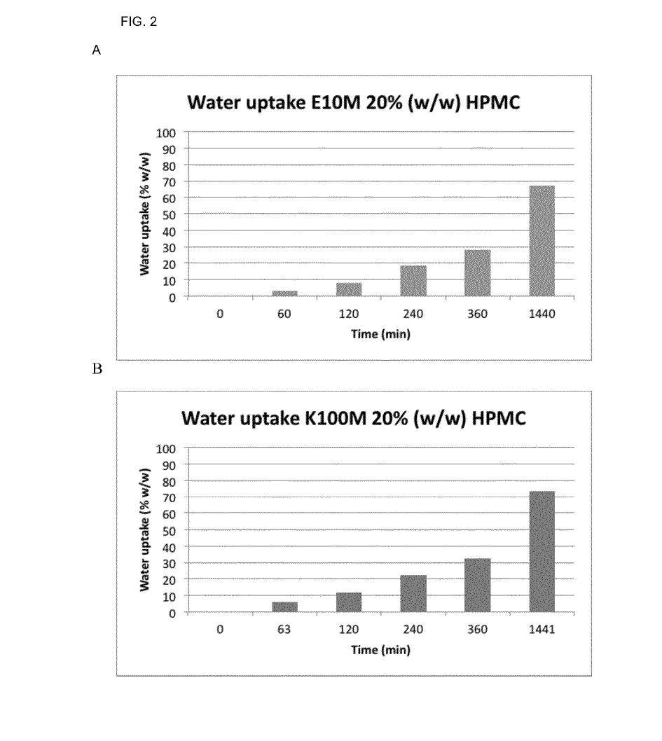

As evident from FIGS. 2a and 2b, for the E-type HPMC polymers E10M and K100M respectively, the drug-loaded inserts obtained using the method of the present invention have a regular water absorption as a function of time. During the first 4 hours no disintegration of the inserts was found, making them particularly interesting for ocular administration.

### 1.2.3. Release Kinetics

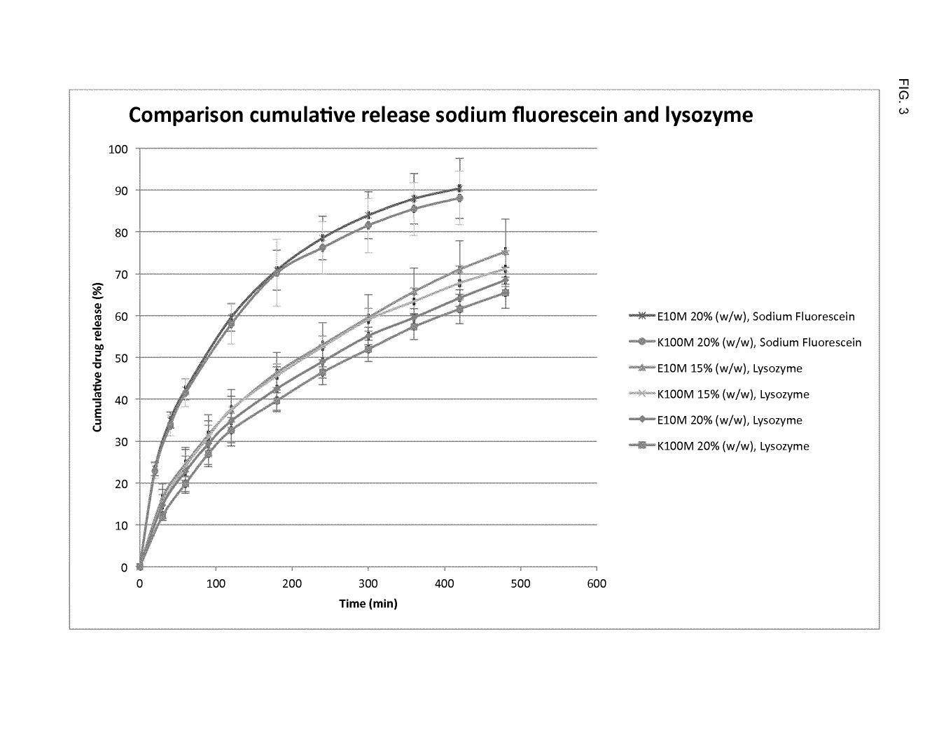

Both Sodium Fluorescein as lysozyme loaded inserts show a steady release profile (see FIG. 3), which is slightly influenced, by the type of HPMC used. The larger lysozyme is released slower when compared to the small molecule (Sodium Fluorecein), but this is likely due to the fact that a larger molecule experiences a higher resistance from the network of the polymer matrix.

### Kinetics

Lysozyme release was found to have a mixed 0 and 1^{st} order kinetic as confirmed in the mathematical model of Peppas-Korsmeyer: according to this model, the logarithm is taken of the cumulative release (y-axis) and the logarithm of the time (x-axis). The slope obtained from the linear regression of this plot is a measure of the kinetics of the release. If the value of the slope n of the equation of the line y=nx+b is smaller than 0.45, then the release from a cylindrical insert follows a first order diffusion. In case the n rico is located between 0.45 and 0.89, this is indicative for mixed 0th and 1st order kinetics. A rico n value greater than 0.89 is an indication of a 0th order kinetics.

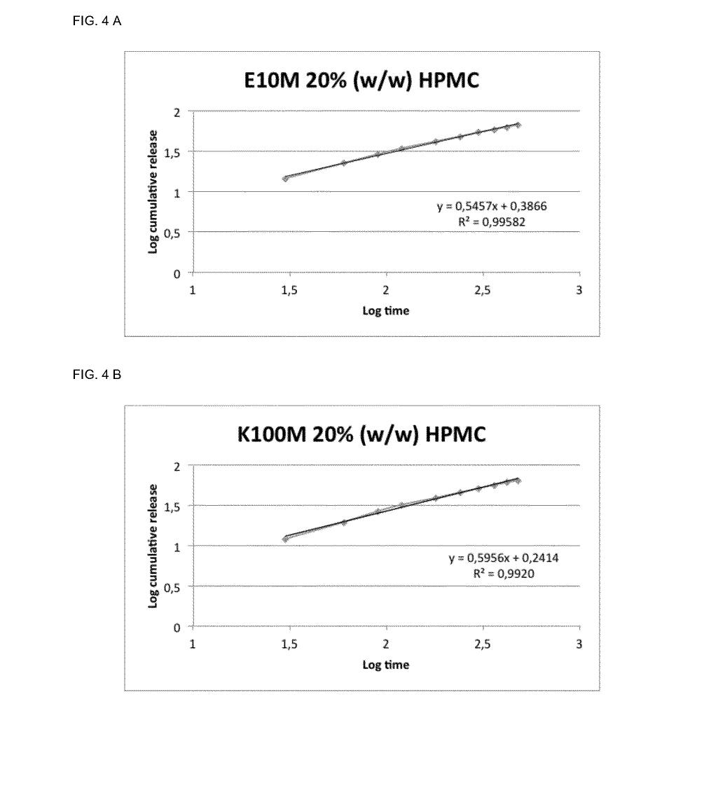

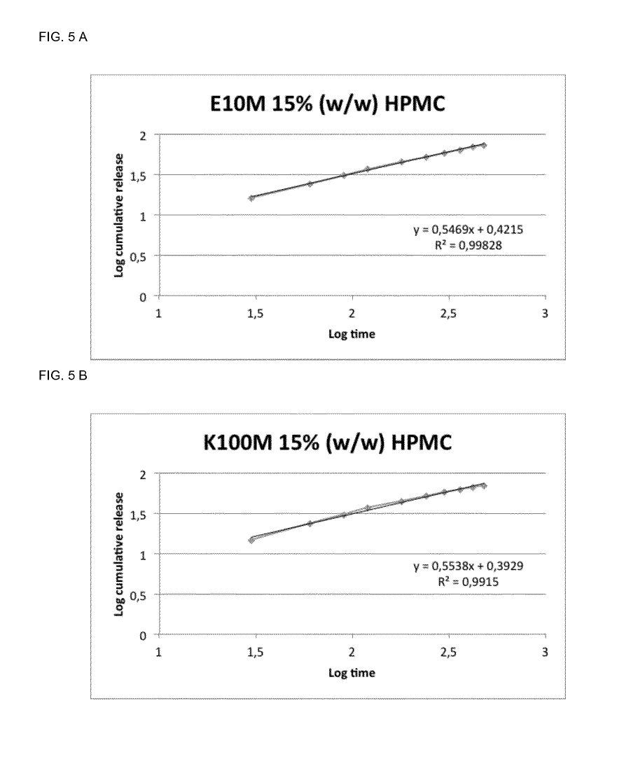

From FIGS. 4a and 4b, the n rico calculated for lysozyme loaded 20% wt E10M and K100M HPMC inserts, equaled 0.5457 and 0.5956 respectively, thus indicative for mixed 0th and 1st order release kinetics. The same mixed 0th and 1st order release kinetics has been confirmed in lysozyme loaded 15% wt E10M and K100M HPMC inserts, showing n rico values of 0.5469 and 0.5538 respectively (see FIGS. 5a and 5b). These experiments thus providing a further functional parameter to confirm that the drug loaded carriers of the present invention comply with the requirements for ophthalmic applications.

As is known to the skilled artisan, ophthalmic inserts are sterile, solid or semi-solid preparations of suitable size and shape, designed to be inserted in the conjunctival sac, to produce an ocular effect. They generally consist of a reservoir of active substance embedded in a matrix or bounded by a rate-controlling membrane. The active substance, to be released over a determined period of time.

### Example 2

### Manufacture of Drug loaded Blister Unit Polymeric Matrices

### 2.A. Methods

Different from the previous example in which only after the rehydration step the drug loaded HPMC matrix extruded in single unit dosage forms, in this example the HPMC polymer hydrogel was prepared and directly poured in blister unit forms. The dehydration step and the rehydration step were accordingly, and directly performed on these blister unit dosage forms.

In this example the E-type HPMC (E10M) has been used as an example, but evidently other HPMC's may be used as well, with in particular the E-type HPMC polymer K100M tested in example 1.

### 2.1.1. Manufacturing Process

Phase 1: HPMC hydrogel was prepared under magnetic stirring at a temperature between 60-100° C. in water. A quantity of 8 PVC blisters (capsule size 4) was filled with the warm HPMC suspension HPMC (550 mg* hydrogel per blister). After cooling, the hydrogel was placed in a refrigerator for at least 2 h at 2° C. The concentration of the hydrogel is 20% wt HPMC. * The final mass of the drug loaded insert is approximately 420 mg.

Phase 2: The resulting HPMC hydrogel was dehydrated at a temperature lower than the gelation temperature, in particular at room temperature, whether or not under a constant air flow rate (for example, in a LAF-cabinet). Different HPMC hydrogels were dried to 30% wt, 50% wt, 75% wt and 100% wt HPMC.

Phase 3: Subsequently, the dehydrated HPMC polymer is charged with a rehydration solution. This solution consists of the API (Sodium Fluorescein, lysozyme or albumin) at a concentration of 3% wt, plasticizer (glycerol) at a concentration of 1 or 5% wt, of water with the addition of HPMC (0-1%). The charging process proceeds at temperatures as close as possible to 0° C. (in practice: in the refrigerator at 2° C.) and at atmospheric pressure. After 24 h or 72 h, the inserts were evaluated for their release profile of the API.

Since each of the HPMC Blister units are completely loaded with the API of the rehydration solution, the homogeneity of the matrix has no bearing on the final concentration of the API in the drug loaded matrix. To assure the desired concentration in the single unit dodge forms in example 1, homogenous distribution of the API in the dehydrated HPMC is a requisite. As such, in this alternative method wherein the HPMC is prepared in single unit dosage forms, composition is easier controlled. It further avoids manipulation (shear forces in the extrusion step of Example 1) of the drug loaded matrix to arrive at the final unit dosage forms.

### 2.1.2. Release Kinetics

### 2.1.2.1. Lysozyme Loaded HPMC Blister Units

The release rate of lysozyme was examined by preparing cumulative release curves for lysozyme and sodium fluorescein for HPMC inserts dehydrated (dried) to 50% wt and dehydrated over 24 h (n=8). Here an insert with a mass of 420 mg was paced in a test tube with 15 g of PBS, and then placed in a non-oscillating hot water bath at 32° C. After 10, 30, 60, 90, 120, 180, 240, 300, 360, 420 and 480 minutes, 2 g samples of the test tube were pipetted and replaced by an equal mass of fresh PBS medium. The absorbance of the sample was measured spectrophotometrically at a wavelength in accordance with the maximum absorption of the drug (278 nm for albumin, 280 nm for lysozyme, 484 nm for sodium fluorescein). In FIG. 6, the amount of released lysozyme and sodium fluorescein at any point of time is expressed as the fraction of the content in relation to the total content present in the insert. As expected, the lysozyme loaded inserts exhibit a significantly lower release profile compared to inserts loaded with sodium fluorescein. (the explanation was already mentioned in the text below 1.2.3 ‘a larger molecule experiences a higher resistance from the network . . . ).

### 2.1.2.2. Albumin Loaded HPMC Blister Units

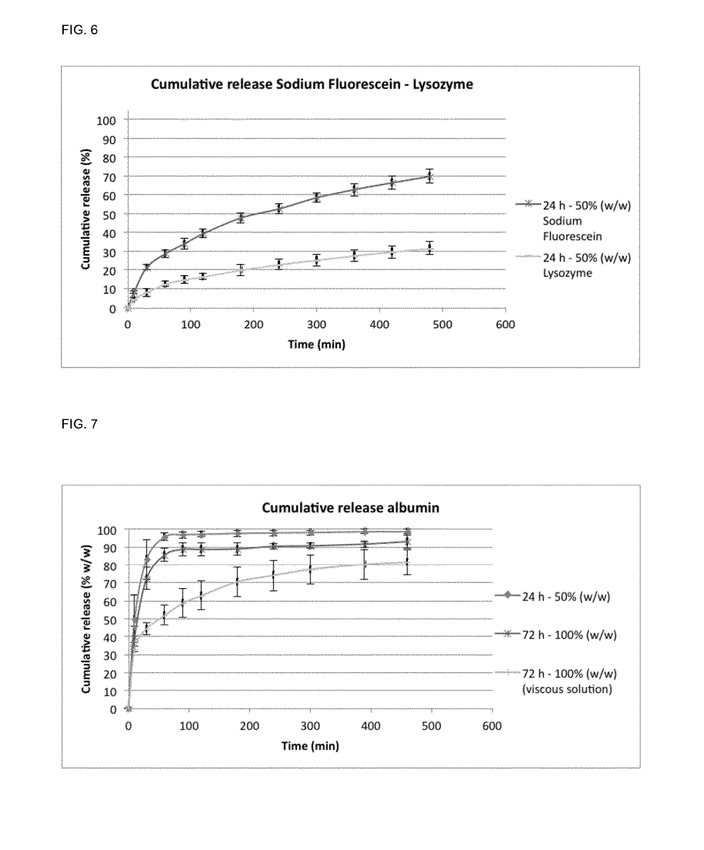

For the Albumin loaded HPMC blisters, different degrees of dehydration were compared. FIG. 7 shows the cumulative release profiles for inserts dehydrated (dried) to 75% wt or 100% wt and dehydrated over 24 h or 72 h (n=8).

Despite the fact that albumin has a higher molecular weight when compared to the smaller lysozyme and thus a slower release from the matrix is expected, the total amount of albumin is released quickly. The high molecular weight of albumin prevents rapid diffusion of albumin molecules in the dehydrated polymer HPMC during the charging process so that most of the molecules will be located close to the surface of the insert. Changing the degree of dehydration of the polymer cylinder or extend the charging time have only a limited impact on the release.

However, changing of the composition of the rehydration solution has proved to have a significant impact on the release speed and release amount of the drug loaded HPMC blister units. By the addition of HPMC in a concentration of 1% m/m to the rehydration solution (=viscous solution, viscous solution), albumin molecules are embedded deeper into the HPMC matrix, so that upon the release of albumin more resistance is provided by the HPMC network with a delayed release effect.

1. A method of manufacturing a drug-loaded carrier suitable for ophthalmic applications, said method comprising:
preparing a hydroxypropyl methyl cellulose (HPMC) hydrogel using standard conditions, but in absence of the drug of interest;
dehydrating the thus obtained HPMC hydrogel to yield a dehydrated HPMC hydrogel that comprises from 25% wt up to 100% wt of said HPMC; and
contacting said dehydrated HPMC hydrogel with a rehydration solution comprising the drug of interest, to obtain said drug loaded carrier;
wherein contacting said dehydrated HPMC hydrogel with the rehydration solution comprising the drug of interest is performed in a temperature range from 0° C. to 25° C., at atmospheric pressure, without exposure to shear forces, without entrapment of air, and without air drying.

2. The method according to claim 1 wherein an ophthalmic drug release formulation is prepared by exposing the drug loaded carrier to the rehydration solution comprising said drug of interest for a time sufficient to allow complete absorption of the rehydration solution.

3. The method according to claim 1, wherein the rehydration solution further comprises a plasticizer.

4. The method according to claim 3, wherein the plasticizer is present in said rehydration solution in an amount up to 50% wt.

5. The method according to claim 3, wherein the plasticizer is selected from glycerol and sorbitol.

6. The method according to claim 5, wherein the plasticizer is glycerol and present in said rehydration solution in an amount of between about 5 to 10% wt.

7. The method according to claim 1, wherein the rehydration solution further comprises HPMC in the range of between about 1 to 15% wt.

8. The method according to claim 1, further comprising shaping the drug loaded carrier.

9. The method according to claim 1, wherein the drug loaded carrier is shaped into rod shaped extrudates.

10. The method according to claim 1 wherein the HPMC hydrogel is selected from E-type, F-type, J-type, K-type or combinations thereof.

11. The method according to claim 10, wherein the HPMC hydrogel consists of E-type HPMC.

12. The method according to claim 10, wherein the HPMC hydrogel consists of K-type HPMC.
